(19) United States
(12) Patent Application Publication
(10) Pub. No.: US 2025/0073263 A1
(43) Pub. Date: Mar. 6, 2025

(54) Method for killing osteosarcoma cells with a bone cement preparation comprising calcium and lithium

(71) Applicant: JOY MEDICAL DEVICES CORPORATION, Kaohsiung (TW)

(72) Inventors: Chien-Ping JU, Kansas City, MO (US); Jiin-Huey Chern LIN, Winnetka, IL (US)

(21) Appl. No.: 18/815,255

(22) Filed: Aug. 26, 2024

Related U.S. Application Data
(60) Provisional application No. 63/579,331, filed on Aug. 29, 2023.

(51) Int. Cl. A61K 33/06; A61K 9/00; A61K 33/42; A61P 35/00

(52) U.S. Cl. CPC A61K 33/06; A61K 9/0012; A61K 33/42; A61P 35/00

(57) ABSTRACT

Use of a bone cement preparation in the fabrication of an anti-osteosarcoma drug for killing osteosarcoma cells is provided, wherein the cement paste preparation contains a solid component having a lithium compound and a calcium compound.

### FIELD OF THE INVENTION

The present invention is related to a treatment of osteosarcoma with a bone cement preparation comprising calcium and lithium, and in particular with a bone cement paste comprising calcium phosphate, calcium sulfate and a lithium compound.

### BACKGROUND OF THE INVENTION

Although adjuvant chemotherapy with extensive surgery treatment is standardized use for osteosarcoma treatment in clinic, however, the prognosis is poor with frequent pulmonary metastasis. Osteosarcoma is radioresistant to standard dose of radiotherapy, so radiotherapy is not used for treating this tumor type. However, while high dose irradiation may be administered after extensive surgery, postoperative radiotherapy wounds normal tissues.

There is a need to kill any possible residual osteosarcoma cells or inhibit growth thereof without adversely affect normal tissues

### SUMMARY OF THE INVENTION

An objective of the present invention is to provide a method for killing osteosarcoma cells with a bone cement preparation after a surgical treatment for removal osteosarcoma from a patient comprising implanting the bone cement preparation in a bone cavity formed by the surgical treatment in said patient, wherein said cement paste preparation comprises a solid component comprising a lithium compound and a calcium compound.

Another objective of the present invention is to provide a novel use of a bone cement preparation in the fabrication of an anti-osteosarcoma drug for killing osteosarcoma cells.

Preferably, the cement paste preparation is the anti-bacterial preparation disclosed in U.S. Pat. No. 10,207,023 B2.

Preferably, the lithium compound comprises lithium carbonate (Li₂CO₃).

### BRIEF DESCRIPTION OF THE DRAWINGS

FIG. 1 shows the results of viability tests of 143 B osteosarcoma cells for the CBCA-400 samples extracted for 24 h, 48 h and 72 h (CBCA-24 hours, CBCA-48 hours, and CBCA-72 hours, respectively), wherein the results of blank tests without CBCA-400 sample extractions are also shown.

FIG. 2 shows the results of viability tests of HOS osteosarcoma cells for the CBCA-400 samples extracted for 24 h, 48 h and 72 h (CBCA-24 hours, CBCA-48 hours, and CBCA-72 hours, respectively), wherein the results of blank tests without CBCA-400 sample extractions are also shown.

FIG. 3 shows the results of viability tests of MG 63 osteosarcoma cells for the CBCA-400 samples extracted for 24 h, 48 h and 72 h (CBCA-24 hours, CBCA-48 hours, and CBCA-72 hours, respectively), wherein the results of blank tests without CBCA-400 sample extractions are also shown.

### DETAILED DESCRIPTION OF THE INVENTION

The bone cement preparation provided according to the present invention comprises a solid component comprising a lithium compound and a calcium compound.

Preferably, the bone cement preparation contains about 5-80%, more preferably about 10-70% of the lithium compound, based on the weight of the solid component.

Preferably, the lithium compound is a lithium salt, lithium oxide, lithium amide (LiNH₂), lithium hydroxide or lithium halide; and more preferably lithium carbonate, lithium sulfate, lithium phosphate, lithium oxide, lithium fluoride, lithium acetate, lithium bromide, lithium hydroxide, lithium nitrate, lithium nitrite, lithium iodide, lithium molybdate (Li₂MoO₄), lithium tetraborate (Li₂B₄O₇), lithium citrate tetrahydrate (Li₃C₆H₅O₇·4H₂O), or lithium stearate (LiC₁₈H₃₅O₂); and most preferably lithium carbonate.

Preferably, the solid component is a powder component comprising the lithium compound and the calcium compound, wherein the calcium compound is selected from the group consisting of a calcium phosphate, calcium sulfate, calcium oxide, calcium carbonate, calcium hydroxide, calcium magnesium phosphate, calcium nitrate, calcium citrate, calcium chloride and a mixture thereof; and more preferably the calcium compound is a calcium sulfate, a calcium phosphate source, or a mixture thereof. Preferably, the calcium phosphate is tetracalcium phosphate (TTCP), dicalcium phosphate, tricalcium phosphate, monocalcium phosphate or a mixture thereof. Preferably, the calcium sulfate is calcium sulfate hemihydrate (CSH), calcium sulfate dehydrate (CSD), anhydrous calcium sulfate, or a mixture thereof.

The bone cement preparation further comprises a setting liquid component with a liquid to powder ratio of 0.20 ml/g to 0.80 ml/g, when the solid component is the powder component.

The present invention also provides a method for treating a subject having osteosarcoma cells comprising forming a bone cement paste by mixing the powder component and the setting liquid component of the bone cement preparation of the present invention; and injecting into a hole or cavity in a bone in said subject with said bone cement paste which sets hard in the hole or cavity in need of said treatment.

Alternatively, the solid component is a block, or granules or pieces obtained by breaking up said block. The block comprises the lithium compound and the calcium compound, wherein the calcium compound is selected from the group consisting of calcium phosphate, calcium sulfate, calcium oxide, calcium carbonate, calcium hydroxide, calcium magnesium phosphate, hydroxyapitite, or a mixture thereof, and preferably is selected from the group consisting of tetracalcium phosphate, dicalcium phosphate, hydroxyapitite, calcium sulfate dihydrate, calcium sulfate hemihydrate, or a mixture thereof.

Preferably, said block is formed by setting said bone cement paste in a mold.

The present invention also provides a method for treating a subject having osteosarcoma cells comprising implanting said block, said granules or said pieces of the bone cement preparation of the present invention in a hole or cavity in a bone in said subject in need of said treatment.

### Experiments

### Materials

CBCA-400 was prepared by mixing appropriate amounts of tetracalcium phosphate (TTCP), dicalcium phosphate (DCPA), calcium sulfate (CSH+CSD) and lithium carbonate (Li₂CO₃) powders at a weight ratio of 30.63:11.43:51.40:6.54.

Hardening (setting) solution used was 0.6M diammonium phosphate (NH₄)₂HPO₄.

CBCA-400 and the hardening solution are available from JOY MEDICAL DEVICES CORPORATION, 1F., No. 63, Luke 2nd Road, Luzhu District, Kaohsiung City, Taiwan.

CBCA-400 was mixed with the hardening solution in a ratio of 1 g:0.36 mL under agitation for one minute, and the resulting paste was shaped into pellets with hands. The pellets were used in experiments immediately after they were prepared.

### Osteosarcoma Cell Viability Test

The cell viability test was performed according to ISO 10993-5. The extraction method was used. An extract was prepared by immersing the pellets in the culture medium at a ratio of 0.2 (g/ml) at 37° C. for 24 h, 48 h and 72 h, respectively, and then collecting the liquid by centrifugation. Osteosarcoma cells in culture medium having a cell concentration of 5×10⁴ cells/ml was incubated in a 96 well microplate (100 μl per well, i.e. 5000 cells per well) in a 5% CO₂ humidified atmosphere at 37° C. After 24 h, the medium was sucked out and then the liquid collected by centrifugation in the above (100 μl) was added to the wells and incubated for 48 h at 37° C. The liquid was sucked out and then a mixture of the culture medium (100 μl) and MTT (10 μl) was added to the wells and incubated for 1 h at 37° C. Cell viability was measured by using the MTT assay. After 1 h incubation, the mixture of medium and MTT was sucked out and 100 μl DMSO was added to each cell of the 96 well microplate and the absorbance (optical density, OD) at 590 nm was measured with Multiskan FC Microplate Photometer.

Three different osteosarcoma cells were used in the test, which are

| | |
|---|---|
| Cell name | MG 63 |

Cell culture medium

| Component | Conc. |
|---|---|
| Minimum Essential Medium powder | 9.6 (g/L) |
| Sodium pyruvate | 0.11 (g/L) |
| Sodium bicarbonate | 1.5 (g/L) |
| Antibiotics-Antimycotic | 10 (ml/L) |
| Fetal Bovine Serum | 100 (ml/L) |

| | |
|---|---|
| Cell name | HOS |

Cell culture medium

| Component | Conc. |
|---|---|
| Minimum Essential Medium powder | 9.6 (g/L) |
| Sodium pyruvate | 0.11 (g/L) |
| Sodium bicarbonate | 1.5 (g/L) |
| Antibiotics-Antimycotic | 10 (ml/L) |
| Fetal Bovine Serum | 100 (ml/L) |

| | |
|---|---|
| Cell name | 143 B |

Cell culture medium

| Component | Conc. |
|---|---|
| Minimum Essential Medium-Alpha | 10.2 (g/L) |
| Medium Powder | |
| Sodium bicarbonate | 1.5 (g/L) |
| 5-Bromo-2′-deoxyuridine, Brdu | 0.0075 (g/L) |
| Antibiotics-Antimycotic | 10 (ml/L) |
| Fetal Bovine Serum | 100 (ml/L) |

### Results

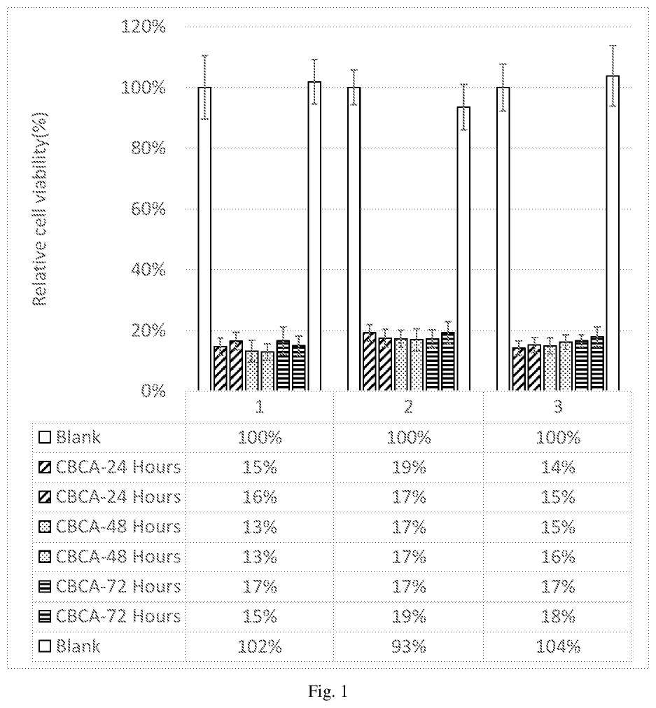

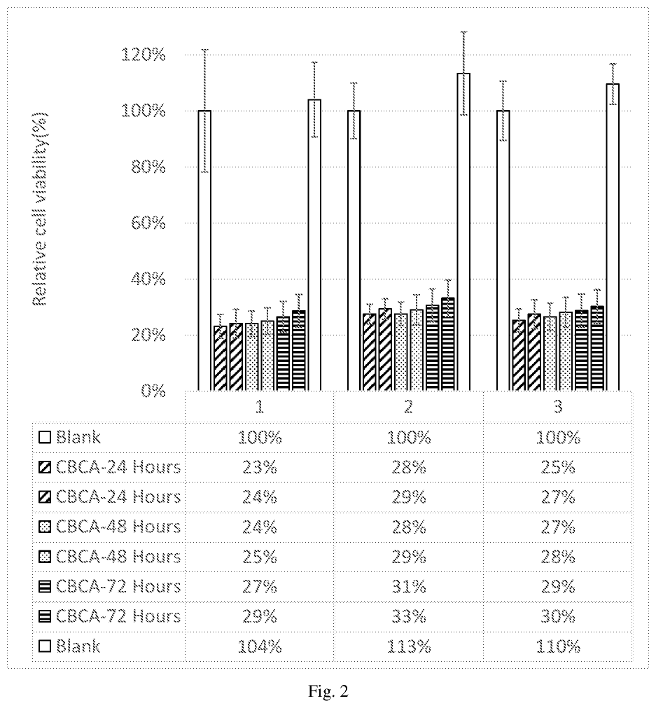

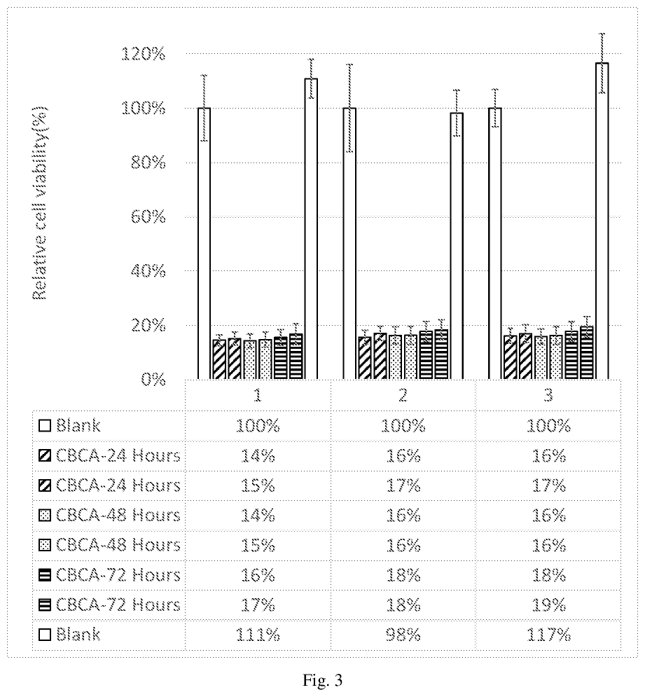

FIG. 1 to FIG. 3 show the results of viability tests of the three different osteosarcoma cells for the CBCA-400 samples extracted for 24 h, 48 h and 72 h (CBCA-24 hours, CBCA-48 hours, and CBCA-72 hours, respectively), wherein the results of blank tests without CBCA-400 sample extractions are also shown.

As shown in FIGS. 1 to 3 the cell viability of the three different osteosarcoma cells are reduced significantly for the 24 h extraction, which stabilizes for the 48 h extraction and the 72 h extraction.

1. A method for killing osteosarcoma cells with a bone cement preparation after a surgical treatment for removal osteosarcoma from a patient comprising implanting the bone cement preparation in a bone cavity formed by the surgical treatment in said patient, wherein said cement paste preparation comprises a solid component comprising a lithium compound and a calcium compound.

2. The method of claim 1 wherein the bone cement preparation contains 5-80% of the lithium compound, based on the weight of the solid component.

3. The method of claim 1 wherein the lithium compound is a lithium salt, lithium oxide, lithium amide (LiNH₂), lithium hydroxide or lithium halide.

4. The method of claim 1 wherein the lithium compound is lithium carbonate, lithium sulfate, lithium phosphate, lithium oxide, lithium fluoride, lithium acetate, lithium bromide, lithium hydroxide, lithium nitrate, lithium nitrite, lithium iodide, lithium molybdate (Li₂MoO₄), lithium tetraborate (Li₂B₄O₇), lithium citrate tetrahydrate (Li₃C₆H₅O₇·4H₂O), or lithium stearate (LiC₁₈H₃₅O₂).

5. The preparation of claim 1 wherein the lithium compound is lithium carbonate.

6. The method of claim 1 wherein the solid component is a powder component comprising the lithium compound and the calcium compound, wherein the calcium compound is selected from the group consisting of a calcium phosphate, calcium sulfate, calcium oxide, calcium carbonate, calcium hydroxide, calcium magnesium phosphate, calcium nitrate, calcium citrate, calcium chloride and a mixture thereof.

7. The method of claim 6 wherein the calcium compound is a calcium sulfate, a calcium phosphate, or a mixture thereof.

8. The method of claim 7 wherein the calcium phosphate is tetracalcium phosphate (TTCP), dicalcium phosphate, tricalcium phosphate, monocalcium phosphate or a mixture, and the calcium sulfate is calcium sulfate hemihydrate (CSH), calcium sulfate dehydrate (CSD), anhydrous calcium sulfate, or a mixture thereof.

9. The method of claim 6 wherein the bone cement preparation further comprises a setting liquid component with a liquid to powder ratio of 0.20 ml/g to 0.80 ml/g, and the powder component and the setting liquid component are mixed to form a paste, wherein said implanting the bone cement preparation in a bone cavity comprises injecting into said bone cavity with said paste which set hard in said bone cavity.

10. The method of claim 1 wherein the solid component is a block, or granules or pieces obtained by breaking up said block, wherein said block is formed by setting said paste as set forth in claim 9.
